(19) United States
(12) Patent Application Publication
(10) Pub. No.: US 2004/0143023 A1
(43) Pub. Date: Jul. 22, 2004

(54) Materials and methods for synthesizing stilbenes

(76) Inventors: John Anthony Hadfield, Salford (GB); Alan Thomson McGown, Manchester (GB); Keira Gaukroger, Hove (GB); Lucy Annette Hepworth, Cheshire (GB); Nicolas James Lawrence, Bristol (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/451,516

(22) PCT Filed: Dec. 14, 2001
(86) PCT No.: PCT/GB01/05534

(30) Foreign Application Priority Data
Dec. 21, 2000 (GB) 0031263.7

(51) Int. Cl. C07C 43/20; A61K 31/075

(52) U.S. Cl. 514/720; 568/660

(57) ABSTRACT

The present invention relates materials and methods for synthesizing stilbenes, and in particular to processes for the synthesis of substituted stilbenes such as combretastatin A4. The present invention relates in particular to methods which are stereoselective for either the cis or the trans isomer of the substituted stilbene, using a Perkin-type condensation of an arylacetic acid and a substituted benzaldehyde, followed by a decarboxylation reaction to produce the substituted cis-stilbenes or a Suzuki-type reaction involving a Z or E-ethenyl halide and a substituted boronic acid in the presence of a palladium catalyst to produce specifically either the Z or E-isomer of substituted stilbenes.

### FIELD OF THE INVENTION

The present invention relates materials and methods for synthesizing stilbenes, and in particular to processes for the synthesis of substituted stilbenes such as combretastatin.

### BACKGROUND OF THE INVENTION

The stilbene cis-combretastatin A-4, isolated from the African bush willow, Combretum caffrum shows exciting potential as an anticancer agent, binding strongly to tubulin and displaying potent and selective toxicity toward tumour vasculature. Cis-combretastatin A-4 is able to inhibit cell growth at low concentrations (IC₅₀, P388 murine leukaemia cell line 2.6 nM). The potency of trans-combretastatin A-4 is much lower and inhibits cell growth in the millimblar range. The isolation of cis-combretastatin A-4 is reported in U.S. Pat. No. 4,996,237 (Arizona Board of Regents).

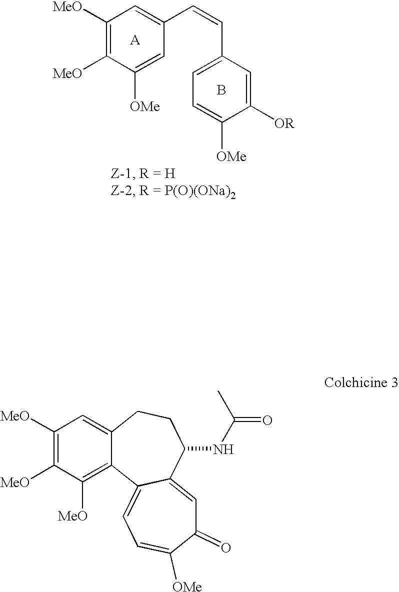

However, the low solubility of cis-combretastatin A-4 in water and saline has led to attempts in the art to make related compounds or prodrugs which retain the activity of cis-combretastatin A-4 as an anticancer agent and which have enhanced solubility. These attempts focus on forming salts or derivatives at the phenolic hydroxyl group of combretastatin. By way of example:

U.S. Pat. No. 5,561,122 (Arizona Board of Regents) which discloses the sodium and potassium salts of cis-combretastatin A-4 and a hemisuccinic acid ester derivative.

WO99/35150 (Arizona Board of Regents) which discloses the lithium, caesium, magnesium, calcium, manganese and zinc salts of cis-combretastatin A-4, and ammonium cation salts with imidazole, morpholine, piperazine, piperidine, pyrazole, pyridine, adenosine, cinchonine, glucosamine, quinine, quinidine, tetracycline and verapamil.

The Wittig reaction is a useful method for the synthesis of combretastatins that has been employed in the past, see for example WO92/16486 (Aston Molecules Limited). However, one significant disadvantage is that a mixture of Z- and E-isomers are produced, thus reducing the yield of the desired Z-isomer. Also, the synthesis of combretastatin A-4 (1) requires five synthetic steps (see FIG. 1) and a difficult chromatographic separation of the two silyl protected stilbene isomers (7,8).

Accordingly, despite the efforts in the art, it remains a significant problem in synthesizing substituted stilbenes such as combretastatin.

### SUMMARY OF THE INVENTION

Broadly, the present invention relates to new methods for producing substituted stilbenes such as combretastatin A4. The present invention relates in particular to methods which are stereoselective for either the cis or the trans isomer of the substituted stilbene.

The present invention therefore provides two methods of synthesis which are stereospecific for cis-stilbenes. In one aspect, the present invention employs a Perkin-type condensation of an arylacetic acid and a substituted benzaldehyde, followed by a decarboxylation reaction to produce the substituted cis-stilbenes. In an alternative aspect, the present invention employs a Suzuki-type reaction involving a Z or E-ethenyl halide and a substituted boronic acid in the presence of a palladium catalyst to produce specifically either the Z or E-isomer of substituted stilbenes. In a further aspect, the present invention provides a method for isomerizing cis-stilbenes to trans-stilbenes, thereby providing a selective method for producing trans-stilbenes from the cis-isomer.

In preferred embodiments of the invention, in addition to the selectivity described above the methods may result in syntheses with reduced numbers of steps as compared to the prior art methods and/or syntheses which employ inexpensive reagents.

Accordingly, in a first aspect, the present invention provides a method for synthesizing a cis-stilbene represented by the general formula:

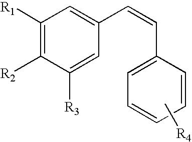

the method comprising:

reacting an arylacetic acid represented by general formula:

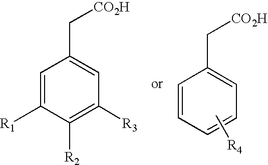

with a substituted benzaldehyde represented by general formula:

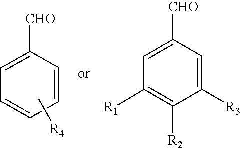

to form a condensation product and decarboxylating the condensation product in the presence of a copper catalyst to produce the cis-stilbene.

Preferably, R₁, R₂ and R₃ are groups which are independently selected from hydrogen, hydroxyl, nitro, amino, aryl, heteroaryl, alkyl, alkoxy, halogen, haloalkyl, NH₂, NHR, NRR′, SR, CONH₂, CONHR, CONHRR′, O-aryl, O-heteroaryl or O-ester, wherein R and R′ are substituted or unsubstituted alkyl groups, e.g. C₁₋₁₀alkyl.

In preferred embodiments, R₁, R₂ and R₃ are independently selected from hydrogen, alkyl, alkoxy, halogen or SR groups, and more preferably, methyl, ethyl, methoxy, ethoxy or fluoro groups.

In the above formula, preferably R₄ is one, two or three substituents at the 2, 3, 4, 5 or 6 positions of the substituted benzaldehyde. In preferred embodiments, preferably the substituents are present at the 4-position, or at the 3-position and the 4-position, or at the 3-position, the 4-position and the 5-position of the benzaldehyde. Where multiple substituents are present they may be the same or different.

Preferably, the R₄ substituent or substituents are independently selected from hydrogen, hydroxyl, nitro, amino, alkyl, alkoxy, halogen, haloalkyl, NH₂, NHR, NRR′, SR, CONH₂, CONHR, CONHRR′, O-aryl, O-heteroaryl or O-ester, wherein R and R′ are substituted or unsubstituted alkyl groups, e.g. C₁₋₁₀ alkyl. In preferred embodiments, the R₄ substituent or substituents are selected from hydrogen, hydroxyl, halogen or alkoxy groups.

For the synthesis of cis-combretastatin-A4, R₁, R₂, and R₃ are all methoxy, and R₄ is a hydroxyl group at the 3-position and a methoxy group at the 4-position.

In all aspects of the present invention, where alkyl or other alkyl containing substituents such as alkoxy are employed, the alkyl groups can be straight chain or branched and are preferably C₁₋₁₀.

This Perkin-type reaction described herein has the advantage that it can be used for the synthesis of cis-combretastatin A-4 in fewer steps that the prior art Wittig reaction and from readily available starting materials. The reaction is also stereoselective and is tolerant of the identity of the R groups present on the arylacetic acid and the substituted benzaldehyde.

Preferably, the initial condensation reaction is carried out in the presence of a carboxylic acid anhydride (either a mixed or symmetrical anhydride) and a tertiary amine. Preferred examples of these reagents are acetic anhydride and triethylamine, added simultaneously or sequentially to the reaction mixture. Typically, the reaction is conducted by heating the reagents under reflux in a solvent, the identity of which can be readily determined by the skilled person. Preferred reaction times are between 1 and 6 hours, preferably about 3 hours. Optionally, the substituted prop-2-enoic acid product is recrystallised prior to continuing to the decarboxylation reaction.

Conveniently, the decarboxylation reaction can be carried out by heating, optionally in the presence of a copper catalyst, typically to a temperature between about 200 and 250° C. Examples of copper catalyst include powdered copper or copper compounds such as copper triflate or copper chromite. The reaction is conveniently carried out in a solvent with a high boiling point such as aromatic pyridine-like bases, e.g. quinoline, substituted quinolines or isoquinolines or similar bases. Preferred reaction conditions for this step involve employing a powdered copper catalyst in a solvent such as quinoline at a temperature of about 230° C.

In some embodiments, the method may comprise the initial steps of synthesizing the arylacetic acid or the substituted benzaldehyde, e.g. using methods available in the literature.

In a further aspect, the present invention provides a method for synthesizing a cis-stilbene represented by the general formula:

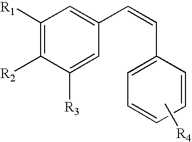

the method comprising

reacting the Z-ethenyl halide represented by the general formula:

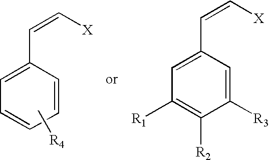

wherein X is a halogen substituent;

with a substituted benzene boronic acid represented by the general formula:

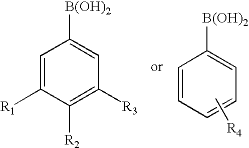

in the presence of a palladium catalyst to produce the Z-stilbene.

In this aspect of the invention, the R₁, R₂, R₃ and R₄ groups are as defined above.

In a further aspect, the present invention provides a method for synthesizing a trans-stilbene represented by the general formula:

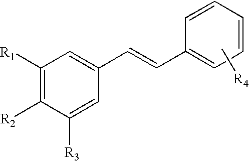

the method comprising

reacting a E-ethenyl halide represented by the general formula:

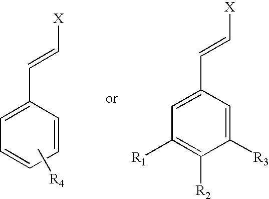

wherein X is a halogen substituent;

with a substituted benzene boronic acid represented by the general formula:

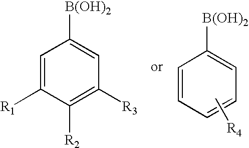

in the presence of a palladium catalyst to produce the E-stilbene.

In this aspect of the invention, the R₁, R₂, R₃ and R₄ groups are as defined above.

In either aspect relating to the Suzuki-type reaction, preferably the Z- or E-ethenyl halide is a bromide or an iodide. Preferably, the palladium catalyst is a palladium(0) catalyst, e.g. tetrakis(triphenylphosphine)palladium(0). Preferred conditions for this reaction include using 1,2-dimethoxyethane as the reaction solvent in the presence of sodium carbonate. However, other conditions (solvents, bases and palladium catalysts) are known in the art and can be used instead of one or more of the preferred reactants or reaction conditions.

The method may comprise the initial step of:

reducing a dihalide ethenyl compound represented by the general formula:

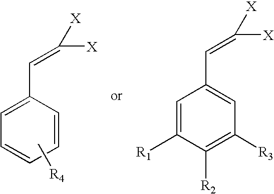

wherein X represents halogen substituents;

to produce a Z-ethenyl bromide.

The reduction reaction may be carried out using a reducing agent such as a tin hydride in the presence of a palladium(0) compound. Preferred conditions employ tributyltin hydride in the presence of tetrakis(triphenylphosphine)palladium(0).

The Suzuki type methodology may also be used to make trans-stilbenes using E-ethenyl bromides and boronic acid, along the lines described above (i.e. same substituent patterns on the reagents etc). E-ethenyl bromides can be synthesized from cinnamic acid and bromines using reactions known in the art.

In any of the above aspects of the invention, the method may comprise the further step of reacting the stilbene (either a Z-stilbene or an E-stilbene) to form a derivative, salt or prodrug. This might be done to improve the properties of compounds used as pharmaceuticals, e.g. to modify their solubility or other pharmacological properties. Preferred salts and derivatives are produced by reaction of the hydroxyl group on the second ring of the stilbene and are discussed in more detail below.

In any of the aspects of the invention, the method may further comprise purifying the stilbene product, or a derivative or salt thereof, and optionally formulating it as a composition, e.g. for pharmaceutical use.

In a further aspect, the present invention provides a method of isomerizing a substituted or unsubstituted Z-stilbene to produce an E-stilbene, the method comprising reacting the Z-stilbene with iodine. In one preferred embodiment, the reaction is carried out around room temperature. A preferred solvent is chloroform, e.g. using 10 mol % iodine. Preferred reaction times are between 15 minutes and one hour. In the examples disclosed herein, this reaction produced trans-CA-4 in virtually quantitative yield (E:Z, 99.8:0.2).

This isomerization reaction can be used separately, or in combination with one of the syntheses of Z-stilbenes described above, to allow the stereoselective production of the corresponding E-stilbene in high yield. However, the reaction is generally applicable and could be employed in other contexts.

Embodiments of the present invention will now be described in more detail by way of example and not limitation with reference to the accompanying drawings.

### BRIEF DESCRIPTION OF THE FIGURES

FIG. 1A shows a prior art synthesis of cis-combretastatin A4 using the Wittig reaction.

FIG. 1B shows the synthesis of Z-stilbenes using a Perkin condensation and decarboxylation reaction.

FIG. 1C shows the synthesis of stilbenes using reactions based on the Suzuki method.

FIG. 1D shows the synthesis of stilbenes using an alternative Suzuki method and an alternative Perkin/decarboxylation method.

### DETAILED DESCRIPTION

Pharmaceutical Compositions

The compounds of the invention may be derivatised in various ways. As used herein “derivatives” of the compounds includes salts, esters such as in vivo hydrolysable esters, free acids or bases, hydrates, prodrugs or coupling partners. In the case of compounds which are combretastatin or analogues thereof, preferably the derivatives are soluble in water and/or saline or can be hydrolysed to provide soluble, and therefore physiologically accessible active agent.

Examples in the prior art of salts or prodrugs of cis-combretastatin A-4 focus on forming salts or derivatives at the phenolic hydroxyl group of combretastatin. These include sodium phosphate salts, sodium and potassium salts (U.S. Pat. No. 5,561,122), lithium, caesium, magnesium, calcium, manganese and zinc salts of cis-combretastatin A-4, and ammonium cation salts with imidazole, morpholine, piperazine, piperidine, pyrazole, pyridine, adenosine, cinchonine, glucosamine, quinine, quinidine, tetracycline and verapamil (WO99/35150).

Salts of the compounds of the invention are preferably physiologically well tolerated and non toxic. Many examples of salts are known to those skilled in the art. Compounds having acidic groups, can form salts with alkaline or alkaline earth metals such as Na, K, Mg and Ca, and with organic amines such as triethylamine and Tris(2-hydroxyethyl)amine. Salts can be formed between compounds with basic groups, e.g. amines, with inorganic is acids such as hydrochloric acid, phosphoric acid or sulfuric acid, or organic acids such as acetic acid, citric acid, benzoic acid, fumaric acid, or tartaric acid. Compounds having both acidic and basic groups can form internal salts.

Esters can be formed between hydroxyl or carboxylic acid groups present in the compound and an appropriate carboxylic acid or alcohol reaction partner, using techniques well known in the art. Examples of esters include those formed between the phenolic hydroxyl of the substituted stilbenes and carboxylic acids, hemisuccinic acid esters, phosphate esters, sulphate esters and selenate esters.

Derivatives which as prodrugs of the compounds are convertible in vivo or in vitro into one of the parent compounds. Typically, at least one of the biological activities of compound will be reduced in the prodrug form of the compound, and can be activated by conversion of the prodrug to release the compound or a metabolite of it. Examples of prodrugs include combretastatin A1 phosphate, combretastatin A4 phosphate and RH1 (2,5, -diaziridinyl-3-(hydroxymethyl)-6-methyl-1,4-benzoquinone.

Other derivatives include coupling partners of the compounds in which the compounds is linked to a coupling partner, e.g. by being chemically coupled to the compound or physically associated with it. Examples of coupling partners include a label or reporter molecule, a supporting substrate, a carrier or transport molecule, an effector, a drug, an antibody or an inhibitor. Coupling partners can be covalently linked to compounds of the invention via an appropriate functional group on the compound such as a hydroxyl group, a carboxyl group or an amino group.

The compounds described herein or their derivatives can be formulated in pharmaceutical compositions, and administered to patients in a variety of forms, in particular to treat conditions which are ameliorated by the activation of the compound.

Pharmaceutical compositions for oral administration may be in tablet, capsule, powder, cream, liquid form or encapsulated by liposomes. A tablet may include a solid carrier such as gelatin or an adjuvant or an inert diluent. Liquid pharmaceutical compositions generally include a liquid carrier such as water, petroleum, animal or vegetable oils, mineral oil or synthetic oil. Physiological saline solution, or glycols such as ethylene glycol, propylene glycol or polyethylene glycol may be included. Such compositions and preparations generally contain at least 0.1 wt % of the compound.

Parental administration includes administration by the following routes: intravenous, cutaneous or subcutaneous, nasal, intramuscular, intraocular, transepithelial, intraperitoneal and topical (including dermal, ocular, rectal, nasal, inhalation and aerosol), and rectal systemic routes. For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, solutions of the compounds or a derivative thereof, e.g. in physiological saline, a dispersion prepared with glycerol, liquid polyethylene glycol or oils.

In addition to one or more of the compounds, optionally in combination with other active ingredient, the compositions can comprise one or more of a pharmaceutically acceptable excipient, carrier, buffer, stabiliser, isotonicizing agent, preservative or anti-oxidant or other materials well known to those skilled in the art. Such materials should be non-toxic and should not interfere with the efficacy of the active ingredient. The precise nature of the carrier or other material may depend on the route of administration, e.g. orally or parentally.

Liquid pharmaceutical compositions are typically formulated to have a pH between about 3.0 and 9.0, more preferably between about 4.5 and 8.5 and still more preferably between about 5.0 and 8.0. The pH of a composition can be maintained by the use of a buffer such as acetate, citrate, phosphate, succinate, Tris or histidine, typically employed in the range from about 1 mM to 50 mM. The pH of compositions can otherwise be adjusted by using physiologically acceptable acids or bases.

Preservatives are generally included in pharmaceutical compositions to retard microbial growth, extending the shelf life of the compositions and allowing multiple use packaging. Examples of preservatives include phenol, meta-cresol, benzyl alcohol, para-hydroxybenzoic acid and its esters, methyl paraben, propyl paraben, benzalconium chloride and benzethonium chloride. Preservatives are typically employed in the range of about 0.1 to 1.0% (w/v).

Preferably, the pharmaceutically compositions are given to an individual in a “prophylactically effective amount” or a “therapeutically effective amount” (as the case may be, although prophylaxis may be considered therapy), this being sufficient to show benefit to the individual. Typically, this will be to cause a therapeutically useful activity providing benefit to the individual. The actual amount of the compounds administered, and rate and time-course of administration, will depend on the nature and severity of the condition being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980. By way of example, and the compositions are preferably administered to patients in dosages of between about 0.01 and 100 mg of active compound per kg of body weight, and more preferably between about 0.5 and 10 mg/kg of body weight.

The compounds may be used in the treatment of cancer and other conditions involving abnormal proliferation of vasculature including diabetic retinopathy, psoriasis and endometriosis.

General

Proton nuclear magnetic resonance (¹H NMR) spectra were recorded on a Brüker AC 300 (300 MHz) or AC 400 (400 MHz) NMR spectrometer. Chemical shifts, δ, for all NMR spectra are given in ppm, relative to tetramethylsilane, and, unless otherwise stated, using CDCl₃ as both solvent and internal standard. Coupling constants (J) were measured in Hz. Melting points were determined on a Gallenkamp melting point apparatus and are uncorrected. The UV/VIS spectra were determined using a Hewlett-Packard HP8452 diode-array spectrophotometer. Extinction coefficients (ε) are presented as their natural logarithms. Microanalyses were carried out by the microanalytical laboratory, Department of Chemistry, University of Manchester. High resolution mass spectroscopy was determined using a Kratos Concept 15 mass spectrometer. Thin layer chromatography (tlc) was performed using precoated aluminium-backed silica gel plates (60 F₂₅₄) with 0.2 mm thickness (Merck), with observation under UV when necessary. Gas chromatography was carried out using an SE 54 column at 195-225 kPa at 1.5 kPa/min. The oven temperature was 180-280° C. at 5° C./min.

1. Stereoselective Synthesis of Z-Stilbenes

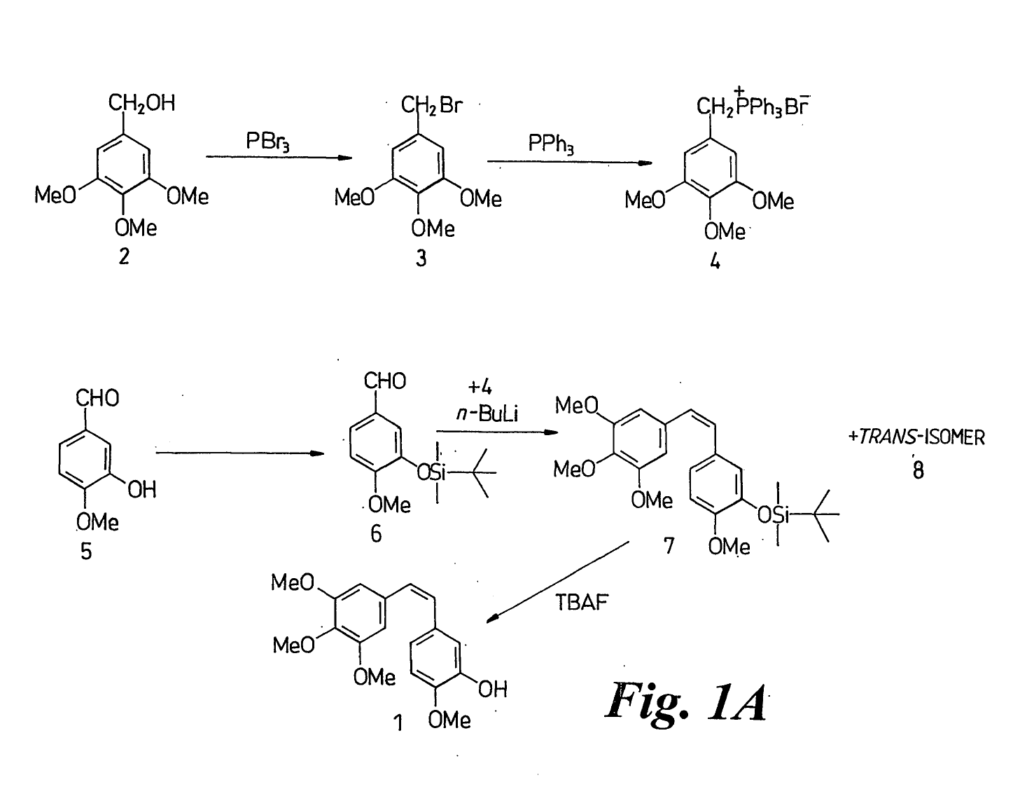

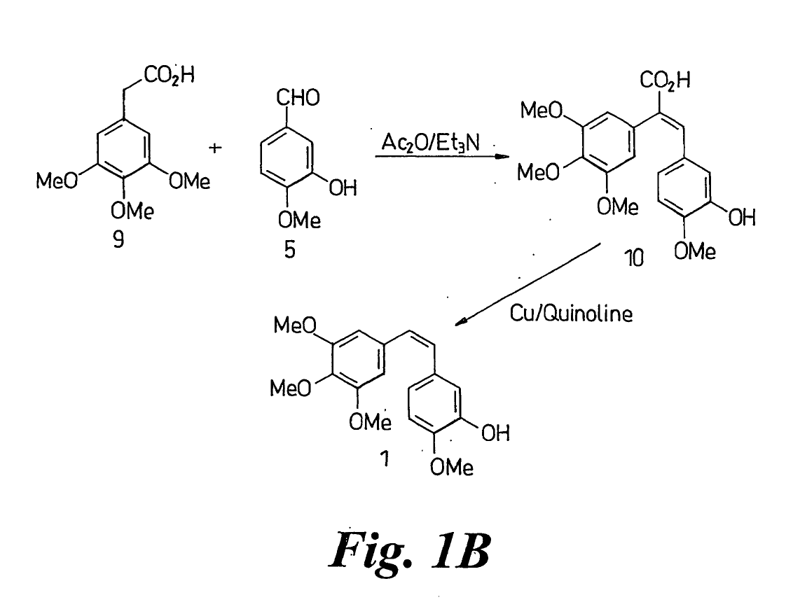

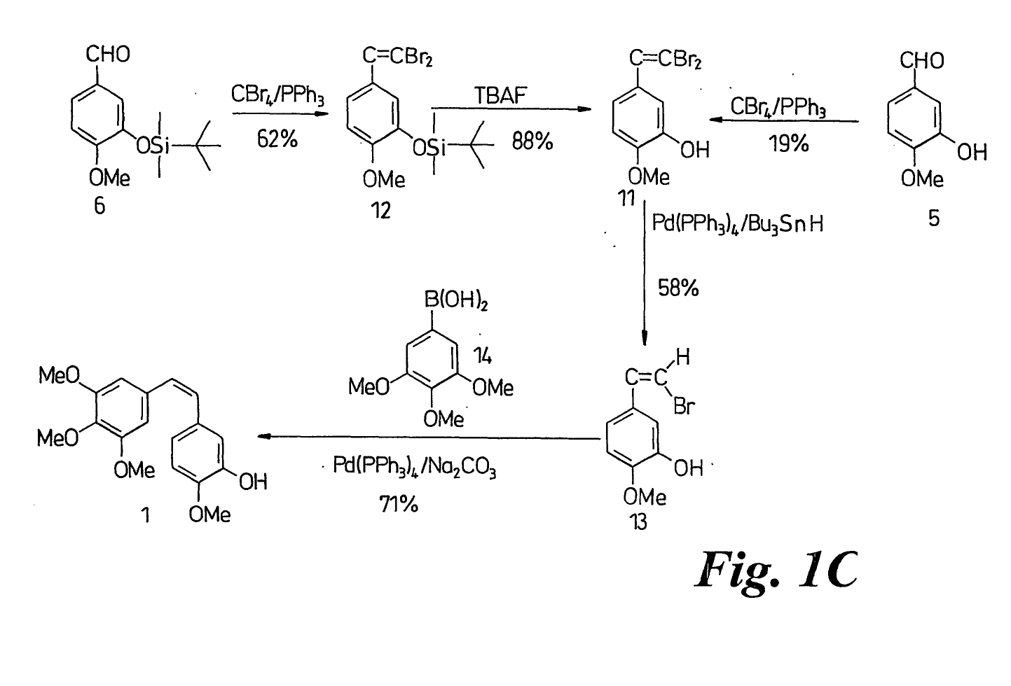

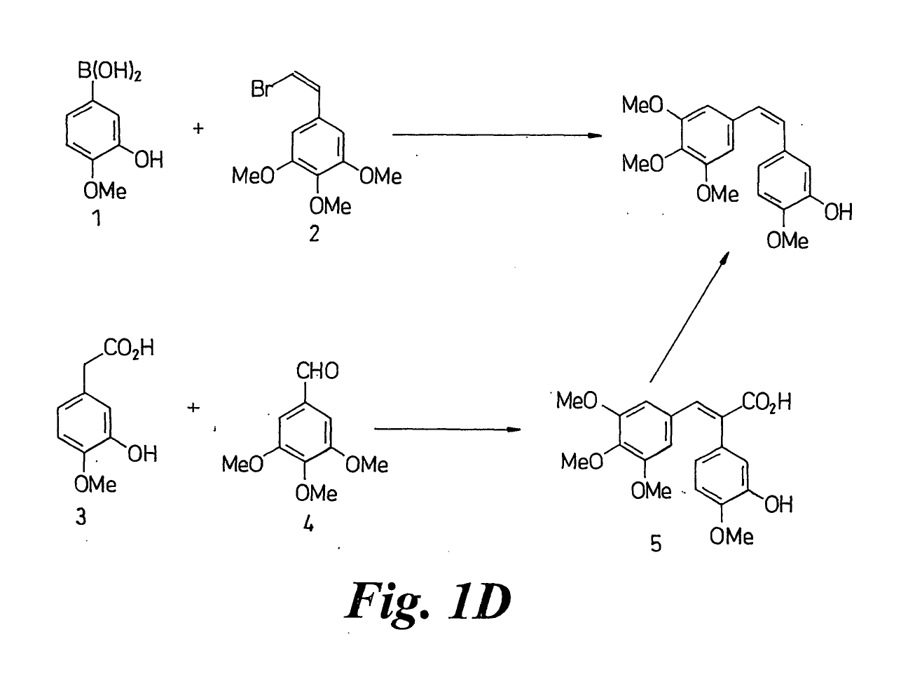

A general method for the synthesis of Z-stilbenes involves the copper-catalysed decarboxylation of an E-2,3-diarylacrylic acid. These acids are prepared by the Perkin-type condensation of an arylacetic acid with a benzaldehyde. However, this two-step methodology of preparing stilbenes has not been applied to the synthesis of combretastatin A-4 (1). In our laboratory the reaction of 3,4,5-trimethoxyphenylacetic acid (9) with 3-hydroxy-4-methoxybenzaldehyde (5) afforded E-3-(3′-hydroxy-4′-methoxyphenyl)-2-(3″,4″,15″-trimethoxyphenyl)prop-2-enoic acid (10) in 60% yield. Decarboxylation of this acid (10) was achieved by heating with copper powder in quinoline at 230° C. The desired combretastatin A-4 (1) was isolated in ca 70% yield (FIG. 1B) after purification by chromatography or by recrystallisation. The overall yield for this two-step synthesis is 41% using inexpensive reagents. The previously published non-stereoselective five-step procedure for the synthesis of combretastatin A-4 (1) yields 31-45% depending on the scale of the reactions.

There are a number of examples in the literature of substituted stilbenes being prepared using Suzuki methodology. This chemistry has been applied to the synthesis of combretastatin A-4 (1).

The first step was to synthesize Z-5-(2′,2′-dibromo-ethenyl)-2-methoxyphenol (11). This dibromo ethenyl compound (11) was synthesized using the Corey-Fuchs Wittig-like bromination of 3-hydroxy-4-methoxybenzaldehyde (5). The yield was poor (ca. 20%) (Fig Section 1a) and so the reaction was repeated with the t-butyldimethylsilyl ether (6) and this gave a higher yield (62%) of the dibromoethene (12). Deprotection of silyl ether (12) to phenol (11) followed by stereoselective reduction of 5-(2′,2′-dibromo-ethenyl)-2-methoxyphenol (11) to the Z-ethenyl bromide (13) was carried out using tributyltin hydride and tetrakis(triphenylphosphine)palladium(0) in 58% yield. Reaction of the Z-bromide (13) with 3,4,5-trimethoxybenzeneboronic acid (14) in 1,2-dimethoxyethane containing sodium carbonate and tetrakis(triphenylphosphine)palladium(0) afforded combretastatin A-4 (1) in a yield of 70%. Starting from benzaldehyde (6) this four-step reaction stereoselectively produces combretastatin A-4 (1) in an overall yield of 23%.

E)-3-(3′-Hydroxy-4′-methoxyphenyl)-2-(3″,4″,5″-trimethoxyphenyl)-prop-2-enoic acid (10)

A solution of 3-hydroxy-4-methoxy-benzaldehyde (5) (0.67 g, 4.4 mmol), 3,4,5-trimethoxyphenylacetic acid (9) (2 g, 8.84 mmol) in acetic anhydride (4 ml) and triethylamine (2 ml) were heated under reflux for 3 h. After careful addition of concentrated hydrochloric acid (6 ml), the resulting solid was filtered off and recrystallised from ethanol to give the title acid (10) (950 mg, 2.63 mmol, 60%) as fine yellow needles. m.p. 237-9° C. δ_{H} (300 MHz d₆-DMSO): 3.68 (6H, s, 2×OCH₃); 3.72 (3H, s, OCH₃); 3.74 (3H, s, OCH₃); 6.44 (2H, s, H-2″, 6″); 6.54 (1H, d, J=1.9, H-2′); 6.61 (1H, dd, J=8.3, 1.9, H-6′); 6.80 (1H, d, J=8.3, H-5′); 7.58 (1H, s, olefinic H).

Z-1-(3′-Hydroxy-4′-methoxyphenyl)-2-(3″,4″,5″-trimethoxyphenyl)ethene—Combretastatin A-4 (1)

(E)-3-(3′-hydroxy-4′-methoxyphenyl)-2-(3″,4″,5″-trimethoxyphenyl)-prop-2-enoic acid (10) (2 g, 5.56 mmol) was added to powdered copper (1.84 g, 28.8 mmol) in quinoline (20 ml, 21.9 g, 0.17 mmol) and the resulting mixture was heated at 200° C. for 2 h. Upon cooling, ether was added and the copper filtered off through celite.

The filtrate was washed with concentrated hydrochloric acid (20 ml) and the aqueous layer was separated and extracted with ether (3×50 ml). The combined organic layers were washed with saturated aqueous sodium carbonate (50 ml), water (2×50 ml), brine (50 ml), dried (MgSO₄) and concentrated in vacuo. Flash column chromatography (petrol/EtOAc 7:3) afforded combretastatin A-4 (1) as a pale yellow crystalline solid (1.19 g, 3.77 mmol, 68%). m.p. 117-8° C. (lit. 116° C.). R_{f}=0.46 (petrol/EtOAc 1:1); δ_{H} (300 MHz): 3.72 (6H, s, 2×OCH₃); 3.68 (3H, s, OCH₃); 3.89 (3H, s, OCH₃); 5.53 (1H, s, OH); 6.42 (1H, d, J=12.4, olefinic H); 6.49 (1H, d, J=12.4, olefinic H); 6.55 (2H, s, H-2″, 6″); 6.75 (1 H, d, J=8.3, H-5′); 6.82 (1H, dd, J=8.3, 1.9, H-6′); 6.94 (1H, d, J=1.9, H-2′).

GC analysis of the crude reaction mixture (without chromatography) showed a ratio of 88:12, cis to trans, but following recrystallisation this changed to 98:2. Analysis following flash column chromatography, without recrystallisation, showed the ratio of cis to trans was 99.4:0.6.

5-(2′,2′-Dibromo-ethenyl)-2-methoxy-phenol (11)

To a well-stirred solution of carbon tetrabromide (12.67 g, 38 mmol) in dichloromethane (80 ml) at 0° C. were added triphenylphosphine (20 g, 76 mmol) and 3-hydroxy-4-methoxybenzaldehyde (5) (5.8 g, 38 mmol). Stirring was continued at 0° C. for 20 minutes and water (80 ml) was added. The aqueous layer was separated and extracted with chloroform (3×25 ml). The combined organic layers were washed with water (2×25 ml) and brine (25 ml), dried (MgSO₄) and concentrated in vacuo. Flash column chromatography (petrol/EtOAc 1:1) afforded phenol (11) as a dark grey solid (2.19 g, 19%). m.p. 94-5° C.; R_{f}=0.20 (CHCl₃); δ_{H} (400 MHz): 3.95 (3H, s, OCH₃); 5.63 (1H, s, OH); 6.87 (1H, d, J=8.3, H-5); 7.06 (1H, dd, J=8.3, 1.9, H-6); 7.25 (1H, d, J=1.9, H-2); 7.39 (1H, s, olefinic H). M⁺, found 305.8888; C₉H₈O₂⁷⁹Br₂ requires 305.8892.

1-(3′-t-Butyldimethylsilyloxy-4′-methoxyphenyl)-2,2-dibromoethene (12)

Dibromoethene (12) was prepared from 3-t-butyldimethylsilyloxy-4-methoxybenzaldehyde (6) (6.5 g, 24.4 mmol) by the method described above for the synthesis of phenol (11). Following flash column chromatography (petrol/EtOAc 20:1) silyl ether (12) was isolated as a pale yellow oil (6.38 g, 62%). R_{f}=0.62 (petrol/EtOAc 9:1); δ_{H} (300 MHz): 0.20 (6H, s, Si(CH₃)₂), 1.02 (9H, s, 3×CH₃), 3.85 (3H, s, OCH₃), 6.85 (1H, d, J=8.3, H-5), 7.11 (1H, dd, J=8.3, 1.9, H-6), 7.23 (1H, d, J=1.9, H-2), 7.38 (1H, s, olefinic H); M⁺, found 420.9830; C₁₅H₂₃O₂Si⁷⁹Br₂ requires 420.9835.

5-(2′,2′-Dibromo-ethenyl)-2-methoxy-phenol (11)

To a stirred solution of 1-(3′-t-butyldimethylsilyloxy-4′-methoxyphenyl)-2,2-dibromoethene (12) (5 g, 11.8 mmol) in dry THF (20 ml) was added tetra-n-butylammonium fluoride (20 ml of 1 M solution, 20 mmol). The resulting yellow solution was stirred for 20 minutes and then treated with water (200 ml). The aqueous layer was separated and extracted with chloroform (3×25 ml). The combined organic layers were washed with water (2×50 ml), brine (50 ml), dried (MgSO₄) and concentrated in vacuo. Following flash column chromatography (petrol/EtOAc 9:1) the phenol (11) was isolated as an orange solid (3.21 g, 10.4 mmol, 88%). m.p. 94-5° C. NMR same as above.

Z-5-(2-Bromo-ethenyl)-2-methoxy-phenol (13)

To a stirred solution of 5-(2′,2′-dibromo-ethenyl)-2-methoxy-phenol (11) (1.5 g, 4.87 mmol) in benzene (40 ml) under argon were added tetrakis(triphenylphosphine)palladium(0) (250 mg, 0.216 mmol), tributyltin hydride (1.3 ml, 4.83 mmol) and pyridine (4 drops). The resulting mixture was stirred overnight and the solvent removed in vacuo. Flash column chromatography (CHCl₃), followed by recrystallisation from 30% aqueous ethanol, afforded Z-5-(2-Bromo-ethenyl)-2-methoxy-phenol (13) as pale yellow crystals (0.65 g, 58%). m.p. 64-6° C.; R_{f}=0.20 (CHCl₃); (Found C, 52.50; H, 6.44; C₉H₉O₂Br requires C, 52.5; H, 6.75%). δ_{H} (300 MHz) 3.94 (3H, s, OCH₃); 5.61 (1H, s, OH); 6.34 (1H, d, J=8.3, olefinic H); 6.87 (1H, d, J=8.3, H-5); 6.98 (1H, d, J=8.3, olefinic H); 7.23 (1H, dd, J=8.3, 1.9, H-6); 7.39 (1H, d, J=1.9, H-2). M⁺, found 227.9782; C₉H₉O₂⁷⁹Br requires 227.9786.

z-1-(3′-Hydroxy-4′-methoxyphenyl)-2-(3″,4″,5″-trimethoxyphenyl)ethene—Combretastatin A-4 (1)

To a stirred solution of Z-5-(2-bromo-ethenyl)-2-methoxyphenol (13) in 1,2-dimethoxyethane (20 ml) under argon was added tetrakis(triphenylphosphine)palladium (0) (76 mg, 0.066 mmol). After 20 min 3,4,5-trimethoxybenzene boronic acid (14) (3.15 mg, 1.49 mmol) and sodium carbonate (138 mg, 1.31 mmol) in water (11.5 ml) were added and the mixture heated under reflux overnight. The aqueous layer was separated and extracted with chloroform (3×20 ml). The combined organic layers were washed with water (2×20 ml), brine (20 ml), dried over magnesium sulphate and concentrated in vacuo. Following flash column chromatography (petrol/EtOAc 7:3) combretastatin A-4 (1) was isolated as a pale yellow crystalline solid (295 mg, 71%). m.p. 117-8° C. (lit. 116° C.); R_{f}=0.46 (petrol/EtOAc 1:1); NMR as above.

2. Synthesis of E-stilbenes using the Suzuki Metholodolgy

The reaction described above using the Suzuki reaction can be adapted to produce E-stilbenes in a stereoselective manner.

E-5-(2-Bromo-ethenyl)-2-methoxy-phenol (15)

To a suspension of finely powdered 3-hydroxy-4-methoxycinnamic acid (4.83 g, 24.9 mmol) in acetic acid (80 ml) was added a solution of bromine (4.05 g, 1.30 ml, 25.4 mmol) in acetic acid (11 ml) dropwise. The acid gradually went into solution and produced HBr. The resulting solution was poured into water (1 l) and the dark pink precipitate filtered off and recrystallised twice from 30% ethanol in water to give 15 as fine white crystals (1.6 g, 28%). m.p. 95-6° C. (lit. 95-6° C.); R_{f}=0.32 (SiO₂ petrol:EtOAc 7:3), δ_{H} (400 MHz) 3.92 (3H, s, OCH₃), 5.63 (1H, s, OH), 6.64 (1H, d, J=13.9, olefinic H), 6.78 (1H, dd, J=8.3, 1.9, H-4), 6.81 (1H, d, J=8.3, H-3), 6.93 (1H, d, J=1.9, H-6), 7.01 (1H, d, J=13.9, olefinic H).

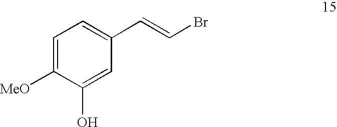

E-1-(3′-Hydroxy-4′-methoxy)-2-(Trimethoxyphenyl)-2-(3″,4″,5″-trimethoxyphenyl)ethene—trans Combretastatin A-4 (16)

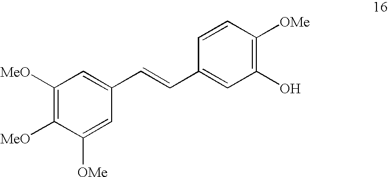

E-5-(2-Bromo-ethenyl)-2-methoxy-phenol (15) (285 mg, 1.24 mmol) and tetrakis(triphenylphosphine palladium)(0) (72 mg, 0.062 mmol) were stirred in 1,2-dimethoxyethane (20 ml) under argon for 20 minutes. 3,4,5-Trimethoxybenzene boronic acid (300 mg, 1.42 mmol) and sodium carbonate (1.31 mg, 1.24 mmol) in water (11 ml) were added and the mixture heated at reflux overnight. The aqueous layer was separated and extracted with chloroform (3×20 ml). The combined organic layers were washed with water (2×20 ml) and brine (20 ml), dried (MgSO₄) and concentrated in vacuo. Flash column chromatography (SiO₂ petrol:EtOAc 7:3) afforded trans-combretastatin A-4 16 (158 mg, 40%). R_{f}=0.41 (SiO₂ petrol:EtOAc 1:1); δ_{H} (300 MHz) 3.88 (3 H, s, OCH₃), 3.94 (9H, s, 3×OCH₃), 5.63 (1H, s, OH), 6.73 (2H, s, H-2″,6″), 6.86 (1H, d, J=8.3, H-5′), 6.89 (1H, d, J=16.2, olefinic H), 6.95 (1H, d, J=16.2, olefinic H), 6.99 (1H, dd, J=8.3, 1.9, H-6′), 7.16 (1H, d, J=1.9, H-2′).

This methodology should be applicable to the synthesis of virtually any trans-stilbene.

3. Isomerisation of Z-Stilbenes as a Practical Route to E-Stilbenes

Having access to this short synthesis of cis-CA-4, renders a simple and selective preparation of trans-CA-4 more viable. When iodine (10 mol %) is added to a solution of cis-CA-4 in chloroform and the resulting mixture is stirred at r.t for 30 mins complete isomerisation occurs, see the scheme below. Following work up, trans-CA-4 is afforded in virtually quantitative yield [E:Z, 99.8:0.2 (determined by GC)].

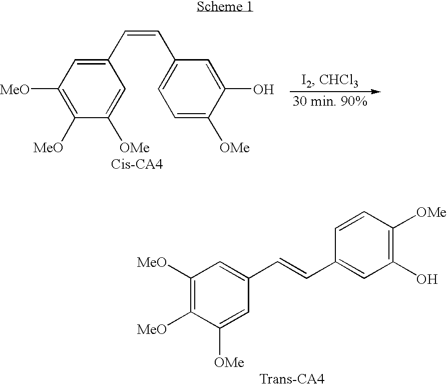

Thus, this aspect of the invention provides a method which can be used alone or in combination with the other syntheses disclosed herein to produce substituted and unsubstituted E-stilbenes.

E-1-(3′-Hydroxy-4′-methoxy)-2-(Trimethoxyphenyl)-2-(3″,4″,5″-trimethoxyphenyl)ethene—trans Combretastatin A-4 (16)

To a solution of cis-Combretastatin A-4 (1) (200 mg, 0.63 mmol) in chloroform (10 ml) was added iodine (16 mg, 0.06 mmol, 10 mol %). The resulting solution was stirred at r.t. for 30 min after which time the solution was washed thoroughly with saturated aqueous sodium metabisulfite, (3×30 ml) to remove the remaining iodine. The yellow solution was then washed with water (2×30 ml), dried (MgSO₄) and concentrated in vacuo to yield the title stilbene 16 (198 mg, 99%) as a viscous yellow oil which solidified on standing. 1δ_{H} (300 MHz, CDCl₃) 3.65 (3H, s, OCH₃), 3.84 (3H, s, OCH₃), 3.90 (6H, s, OCH₃), 5.58 (1H, s, OH), 6.70 (2H, s, H-2″ and H-6′″), 6.81 (2H, d, J=8.3 Hz, H-5′), 6.85 (1H, d, J 16.6 Hz, CH═C), 6.90 (1H, d, J 16.6 Hz, CH═C) 6.99 (1H, dd, J 8.3 Hz, J=2.0 Hz, H-6′) 7.15 (1H, d, J=2.0 Hz, H-2′).

The reference mentioned herein are all expressly incorporated by reference.

1. A method for synthesizing a cis-stilbene represented by the general formula: 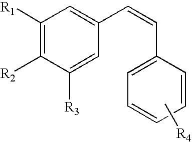
the method comprising:
reacting an arylacetic acid represented by general formula: 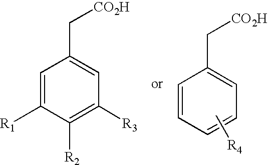
with a substituted benzaldehyde represented by the general formula: 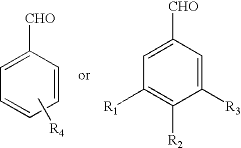
to form a condensation product and decarboxylating the condensation product in the presence of a copper catalyst to produce the cis-stilbene;
wherein the R₁, R₂ and R₃-groups are independently selected from hydrogen, alkyl, alkoxy, halogen or SR, where R is a substituted or unsubstituted alkyl group; and
R₄ is a substituent or substituents at the 4-position, or at the 3-position and the 4-position, or at the 3-position, the 4-position and the 5-position; and
the R₄ substituent or substituents are selected from hydrogen, hydroxyl, halogen or alkoxy groups.

2. The method of claim 1, wherein R₁, R₂ and R₃ are independently selected from methyl, ethyl, methoxy, ethoxy or fluoro groups.

3. The method of claim 1 or claim 2, wherein for the synthesis of cis-combretastatin-A4, R₁, R₂, and R₃ are all methoxy, and R₄ is a hydroxyl group at the 3-position and a methoxy group at the 4-position.

4. The method of any preceding claims, wherein the reaction to produce the condensation product is carried out in the presence of a carboxylic acid anhydride and a tertiary amine.

5. The method of claim 4, wherein the carboxylic acid anhydride and the tertiary amine are acetic anhydride and triethylamine, added simultaneously or sequentially to the reaction mixture.

6. The method of any preceding claims, wherein the reaction to produce the condensation product is conducted by heating the reagents under reflux in a solvent.

7. The method of claim 6, wherein the reaction time is between 1 and 6 hours.

8. The method of any one of the preceding claims, wherein the condensation product is is recrystallised prior to carrying out the decarboxylation reaction.

9. The method of any one of the preceding claims, wherein the decarboxylation reaction is carried out by heating to a temperature between about 200 and 250° C.

10. The method of any one of the preceding claims, wherein in the decarboxylation reaction, the condensation product is heated in the presence of the copper catalyst.

11. The method of claim 10, wherein the copper catalyst is powdered copper or a copper compound such as copper triflate or copper chromite.

12. The method of any one of claims 9 to 11, wherein the decarboxylation reaction is carried out in a high boiling point solvent.

13. The method of claim 12, wherein the high boiling point solvent is a quinoline, a substituted quinoline or an isoquinoline.

14. The method of any one of the preceding claims, wherein the decarboxylation reaction uses a powdered copper catalyst in a solvent such as quinoline at a temperature of about 230° C.

15. The method of any one of the preceding claims, which comprises the initial step of synthesizing the arylacetic acid or the substituted benzaldehyde.

16. The method of any one of the preceding claims, further comprising the step of reacting the stilbene (either a Z-stilbene or an E-stilbene) to form a derivative, salt or prodrug.

17. The method of any one of the preceding claims, further comprising purifying the stilbene or a derivative or salt thereof.

18. The method of claim 17, further comprising formulating the stilbene in a pharmaceutical composition.
